(19) United States
(12) Patent Application Publication
(10) Pub. No.: US 2019/0009241 A1
(43) Pub. Date: Jan. 10, 2019

(54) Mobile Solid Phase Reaction System and Method

(71) Applicant: Swedish Biomimetics 3000 Limited, Hethel (GB)

(72) Inventor: Andrew Richard Russell PREWER, Hethel (GB)

(21) Appl. No.: 16/069,001

(22) PCT Filed: Jan. 11, 2017
(86) PCT No.: PCT/GB2017/050064

§ 371 Date: Jul. 10, 2018

(30) Foreign Application Priority Data
Jan. 11, 2016 (GB) 1600464.0

(51) Int. Cl. B01J 19/00; C07K 1/04; C07H 1/00; C07H 21/00; C07H 3/06

(52) U.S. Cl. CPC B01J 19/0046; C07K 1/045; C07H 1/00; C07H 21/00; C07H 3/06; B01J 2219/00871; B01J 2219/00596; B01J 2219/00605; B01J 2219/00725; B01J 2219/00722; B01J 2219/0059

(57) ABSTRACT

A system and method are disclosed. A system for contacting a mobile solid phase with a flowing fluid phase includes: one or more reaction module, wherein the one or more reaction module comprises a conduit for the passage of a fluid phase and a solid phase, the conduit comprising a fluid input port and a fluid outlet port, and a first service module operably connected to a first side of a reaction module, the first service module for supplying and/or receiving the fluid phase to and/or from the reaction module, wherein the system is configured for passing a solid phase through the reaction module, via the conduit.

The present invention relates to a system and method. In particular, but not exclusively, the present invention relates to apparatuses, systems and processes for performing treatments involving a mobile solid phase and a mobile fluid phase.

### BACKGROUND

Solid phase synthetic methods have been extensively used in the preparation of a wide variety of compounds.

There will first be described prior art describing the chemistry of solid phase synthesis.

A useful review of the preparation of cellulose-bound peptide arrays is Hilpert K et al, Cellulose-bound peptide arrays: Preparation and applications, Biotechnol. Genet. Engineer. Rev. 2007, 24:31-106. Hilpert et al teach that cellulose is a polysaccharide with free hydroxy groups and that, since these hydroxy groups are less reactive than amino groups, the direct attachment of amino acids often leads to low yields. To make the cellulose suitable for the synthesis of peptides, the cellulose surface is modified to change the functionalisation from hydroxy to amino groups. It is further taught that modification of the cellulose often involves insertion of a spacer molecule permitting better access to the amino groups on the cellulose. After functionalisation, the amino acids are taught to be coupled either as an active ester (e.g. pentafluorophenyl ester) solution or as in situ activated mixtures. In situ activation is described as mostly carried out with DIC (N, N′-diisopropyl carbodiimide) and HOBt (N-hydroxybenzotriazole) shortly before coupling. Pages 34-42 of Hilpert et al are referred to here in particular as describing pre-treatment of the cellulose and peptide synthesis. Techniques for screening peptide arrays are described later in the same paper. Hilpert et al mention also non-cellulosic substrates (on page 33) and the synthesis of non-peptidic compounds (on page 43).

Mutulis F et al, J. Comb. Chem. 2003, 5:1-7 describe a method for producing non-random peptide libraries using cotton discs. The discs were activated in (25 v/v % in DCM) TFA (to protonate the hydroxy groups of the cotton). To enable peptide synthesis a handle was attached to the cotton to provide access to reagent molecule and a linker was then attached to the handle to provide a reactive site for Fmoc solid phase synthesis. The handle was 6-aminocaproic acid (H₂N—(CH₂)₅—COOH) and the linker was Fmoc Rink linker 4-[(2,4-dimethoxyphenyl)(Fmoc-amino)methyl]-phenoxyacetic acid. Peptides having different amino acid sequences were then synthesised on different discs.

The synthesis of oligonucleotide arrays on cellulose is described by Frank W et al, Nucl. Acids. Res. 1983, 11:4365-4377. Paper discs were pretreated by coupling protected nucleoside-3′-succinates were coupled to the discs by condensation of their carboxylic functions with the hydroxy groups of the cellulose in the presence of MSNT (1-(mesitylene-sulfonyl)-3-nitro-1,2,4,-triazole). After deprotection, a dimethoxy-tritylated base protected phosphodiester is coupled to the pretreated paper disc and further dimethoxy-tritylated base protected phosphodiester building blocks are linked step by step to form the completed oligonucleotide.

Fromont C et al, Chem. Commun. 2000, 283-284 describes the use of triple branching symmetrical dendrimers to increase the loading of a solid phase in the form of resin beads. The authors describe the synthesis of a tri-branching symmetrical dendrimer on the solid phase with an 18-fold amplification of loading. The tri-functional dendrimer monomers were prepared in bulk by alkylation of tris with acrylonitrile followed by nitrile hydrolysis in a saturated solution of HCl in dry MeOH to give the methyl ester. The hindered amino group of the methyl ester was converted to the corresponding isocyanate by treatment with Boc₂O and DMAP as described by Knölker to give a stable symmetrical monomer (Knölker H-J et al, Angew. Chem., Int. Head. Engl. 1995, 34: 2497) an amino methyl polystyrene resin was directly derivatised with the isocyanate. The methyl ester was displaced by propane-1,3-diamine. The process was repeated to give Generation 2.0 dendrimer beads. The use of glass as a substrate for attachment of analytes or biological molecules is well known. For example, Millipore Data Sheet “DNA Nucleoside Controlled Pore Glass (CPG®) media” describes the use of DNA-CPG products for the solid phase synthesis of oligonucleotides using phosphoramidite chemistry. The data sheet is identified as Lit. No. DS0010EN00 Rev. A 03/06.

Shenoy N R et al, Protein Sci. 1992, 1: 58-67 describes the use of carboxylic acid-modified polyethylene as a solid phase support for polypeptides. The peptides are attached by coupling the N-terminal amino group of the peptides to the activated carboxyl groups of the film. The carboxylic acid-modified polyethylene (PE-COOH film) was provided by the Pall Corporation of Long Island, N.Y. The highest yields of covalently attached peptide were obtained when 1,3-dicyclohexylcabrodiimide (DCC) was used as an activating agent.

It is also known to use so-called “CLEAR” resins (Cross-Linked Ethoxylate Acrylic Resin) as supports for solid phase peptide synthesis. Such CLEAR products are described in U.S. Pat. Nos. 5,910,554 and 5,656,707 and are produced by Peptides International, Inc.

Sanghvi Y S et al, Pure and Applied Chemistry, 2001, 73: 175-180 describe reusable solid support chemistries for oligonucleotide synthesis. The reusable solid support technology is based on the use of a hydroquinone diacetic acid spacer arm between the 3′-end of the first nucleoside and the hydroxyl-functionalised support. Details of the chemistry have been published in Pon R T et al, Nucleic Acids Research, 1999, 27: 15-31.

For a review article relating to developments in solid phase synthesis supports see Sucholeiki, Molecular Diversity, 1999, 4: 25-30. The new solid phase synthesis supports described include cross-linked polyoxyethylene-polystyrene and polyoxyethylene-polyoxypropylene and polyamidoamine dendrimers attached to TentaGel support.

The solid phase PEGylation of a protein has been described by Lee B K et al in Bioconjugate Chem., 2007, 18: 1728-1734. Recombinant interferon α-2a was absorbed to a cation exchange resin and PEGylated at the N-terminus by mPEG aldehydes through reductive alkylation using NaBH3CN as reducing agent.

An increasingly important class of polymer is organic semiconductor polymers. Turner D et al, Mat. Res. Soc. Symp. Proc., 2003, 771: L8.8.1-L8.8.5 describe a solid phase synthetic strategy for the production of organic semiconductors. The strategy uses a germanium-based linker and Suzuki-type cross-coupling protocols and has been demonstrated for the iterative synthesis of both a regio-regular oligo-3-alkylthiophene and an oligoarylamine. Turner et al is included herein in its entirety for all purposes, as are references 1, 2, 3 and 4 of Turner et al.

For further information on solid phase synthesis techniques, reagents and substrates see Organic Synthesis on Solid Phase: Supports, Linkers, Reactions, Florencio Zaragoza Dörwald, Wiley-VCH, Second Edition, 2002, ISBN 352730603X.

There will be described next prior art relating to methods performed using a mobile solid phase.

EP0385433A2 discloses a method and apparatus for continuous synthesis on a solid carrier. The solid carrier, for example in the form of a band or thread, has functional groups and is passed through successive reaction and processing zones in a sequence corresponding to the reaction and processing steps of the synthesis concerned. The reaction and processing zones are in the form of baths of liquid, and the carrier coming from the preceding synthetic step is pressed between a pair of rollers to remove most of the liquid from the preceding step.

EP1304162A2 discloses a method and apparatus for the preparation of polymer arrays on the surface of a flexible elongate web. The apparatus includes a dispensing head and optionally other treatment stations including reagent baths and water baths, the latter being for rinsing. A detection station may be included for detecting fluorescence. The web is driven through these various stages of the apparatus for successive treatments to be carried out at successive stages.

US2002/0001544A1 discloses a system and method for high throughput processing of droplets. The droplets are dispensed onto a moving surface from one or more reagent addition stations through which the moving surface moves. A combinatorial synthesis may be accomplished and assays can be performed directly on the chemical reaction products on the moving surface.

WO2009/004344 discloses a solid phase reaction method and apparatus including passing an elongate material through at least one reaction zone and reacting the substance in the zone.

WO2009/004344 discloses a solid phase reaction method and apparatus including an elongate material with a substance provided thereon through at least one reaction zone and reacting the substance in the zone. The elongate material may be passed through a plurality of reaction and rinsing zones, and data may be collected from the elongate material by testing apparatus. It is described that the reaction zone is preferably a conduit. The conduit may be defined by apparatus including three plates engaging one another in face-to-face arrangement such that a continuous conduit is formed including conduits formed in each end plate and an aperture through the central plate. Inlets/outlets are provided for the elongate material to enter and exit the reaction zone from the sides of the apparatus. In this way, the elongate material may continue to a rinsing zone or another reaction zone adjacent the first reaction zone in series. Fluid access holes are provided for fluid to access the reaction zone from the front of the apparatus (i.e. at an angle perpendicular to the entry of the elongate material). In this way different fluids can access different zones in parallel. Initial assembly requires an elongate material (e.g. ribbon) to be threaded into the apparatus and through each section of each reaction zone by hand. The remainder of the apparatus is then built around the sets of plates.

All the above prior art documents are included herein by reference in their entirety for all purposes.

The disclosure comprises in particular improvements and/or modifications of the disclosure of WO2009/004344.

According to a first aspect of the present invention there is provided a system for contacting a mobile solid phase with a flowing fluid phase, comprising:
one or more reaction module,
   wherein the one or more reaction module comprises a conduit for the passage of a fluid phase and a solid phase, the conduit comprising a fluid input port and a fluid outlet port, and
a first service module operably connected to a first side of a reaction module, the first service module for supplying and/or receiving the fluid phase to and/or from the reaction module,
wherein the system is configured for passing a solid phase through the reaction module, via the conduit.

According to a second aspect of the present invention there is provided a reaction module for contacting a mobile solid phase with a flowing fluid phase, comprising:
a conduit for the passage of a fluid phase and a solid phase, the conduit comprising a fluid input port and a fluid outlet port;
wherein the fluid input port is provided in a first side of the reaction module, and the fluid outlet port is provided in a further side of the reaction module,
wherein the reaction module is configured to be operably connectable to a first service module, and a further service module or a further reaction module on the further side of the reaction module, for providing the fluid phase and the solid phase to the reaction module, and
wherein the reaction module is configured for passing an elongate solid phase there through, via the conduit.

According to a third aspect of the present invention there is provided a service module for providing services to a reaction module for contacting a mobile solid phase with a flowing fluid phase, the service module comprising:
a housing having a side for operable connection with a reaction module,
the housing comprising a conduit for flowing fluid, the conduit having a first end for connection to a fluid supply or fluid drain, and a second end at the side of the housing for connection with a conduit in the reaction module,
wherein the service module is configured for providing one or more services to the reaction module.

According to a fourth aspect of the present invention there is provided a method of performing a solid state synthesis using the system of the first aspect, the method comprising:
passing the mobile solid phase from the first service module to a further reaction module or service module, via the conduit of the reaction module; and
causing a fluid phase to enter the conduit of the reaction module through a first input port, flow through the conduit and leave through a first fluid output port.

Certain embodiments of the invention provide the advantage that a system having a series of reaction zones for a chemical process can be provided with relative ease of assembly and use compared to known methods and apparatuses.

For example, an extended process line comprising multiple process stages can be readily assembled by connecting a number of service and reaction modules. Any single reaction stage can be extended (corresponding to an extended reaction time) through the connection of multiple reaction modules for performing a single reaction, or through the use of modules and service modules to perform the same synthetic or other process step.

Certain embodiments of the invention provide the advantage that service elements can be separated from the reaction zone, and a system can be provided with a higher degree of flexibility to the user.

Certain embodiments of the invention provide the advantage that a system is provided for contacting a solid phase with a fluid phase that is more flexible in terms of set up and use compared to known arrangements.

For example, a number of modules may be connected to form a system with minimum tool requirements and a reduced number of operations compared to known arrangements.

Other advantages are set out below.

Embodiments of the invention are further described hereinafter with reference to the accompanying drawings, in which:

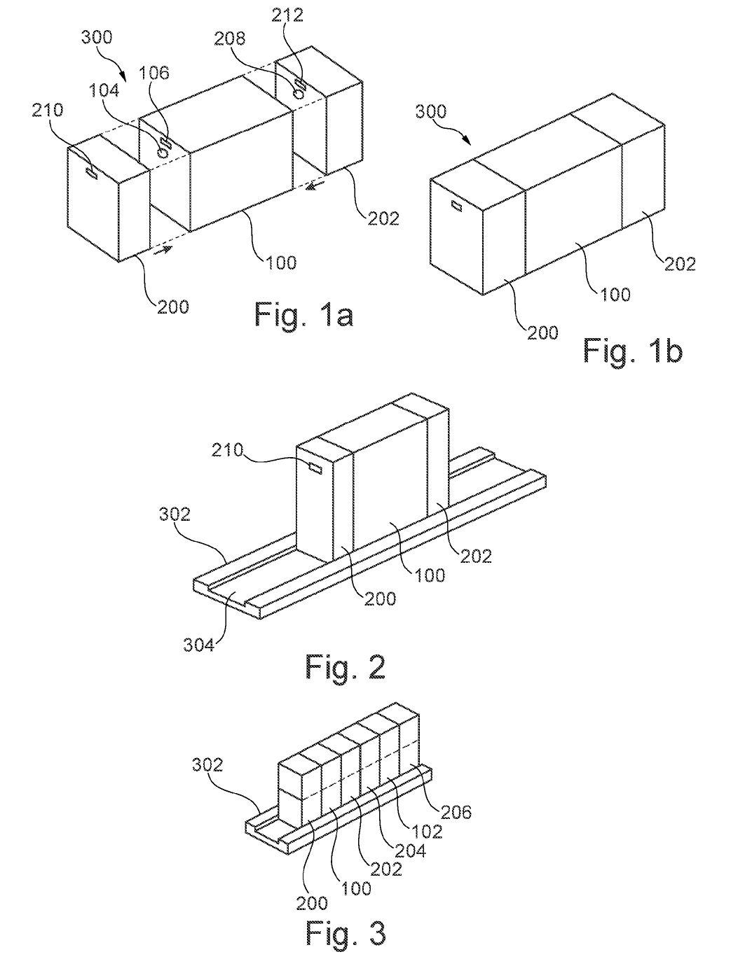

FIG. 1 illustrates a system for contacting a solid phase with a fluid phase;

FIG. 2 illustrates the system of FIG. 1 with further elements;

FIG. 3 illustrates the system of FIG. 1 with yet further elements;

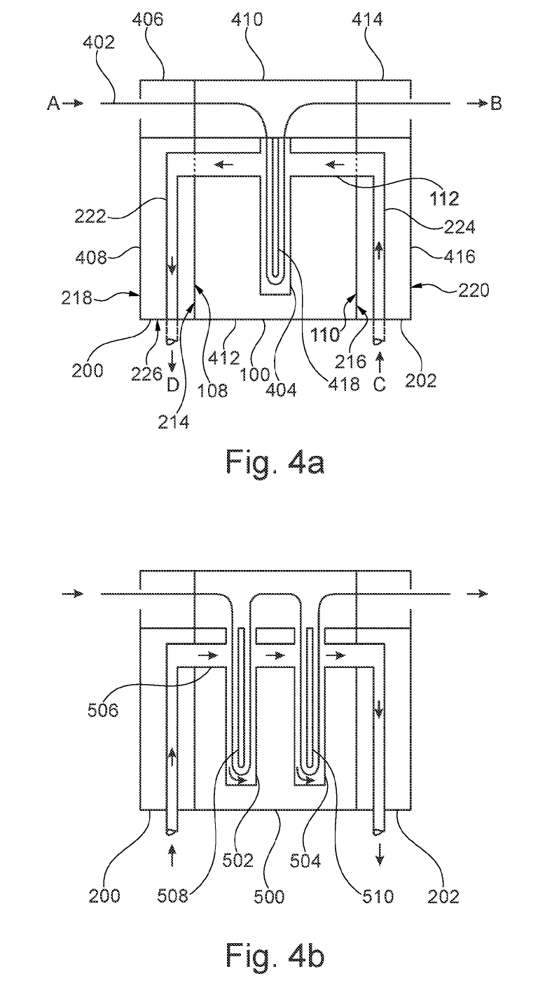

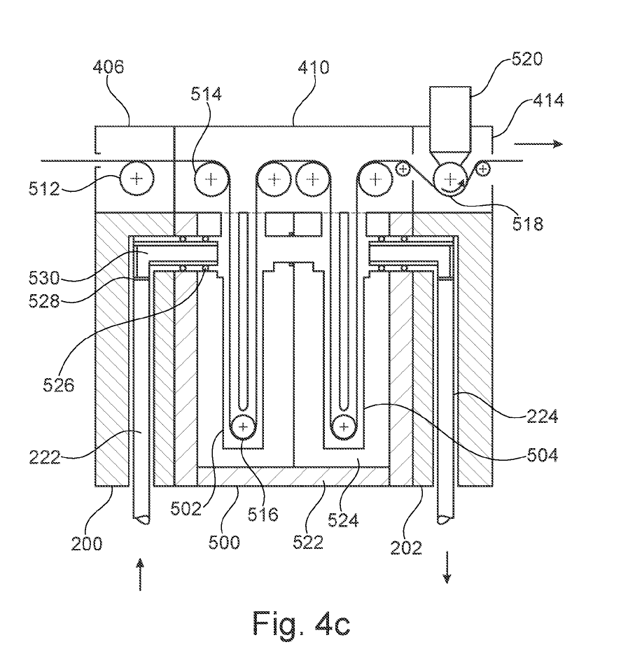

FIGS. 4a, 4b and 4c illustrate a cross section through an assembly;

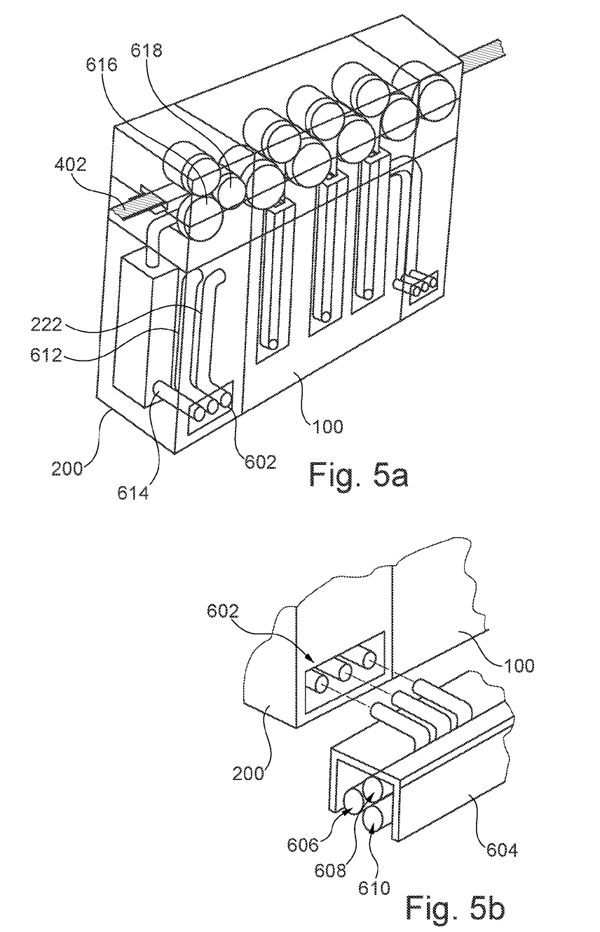

FIGS. 5a and 5b illustrate a system and a service ducting module;

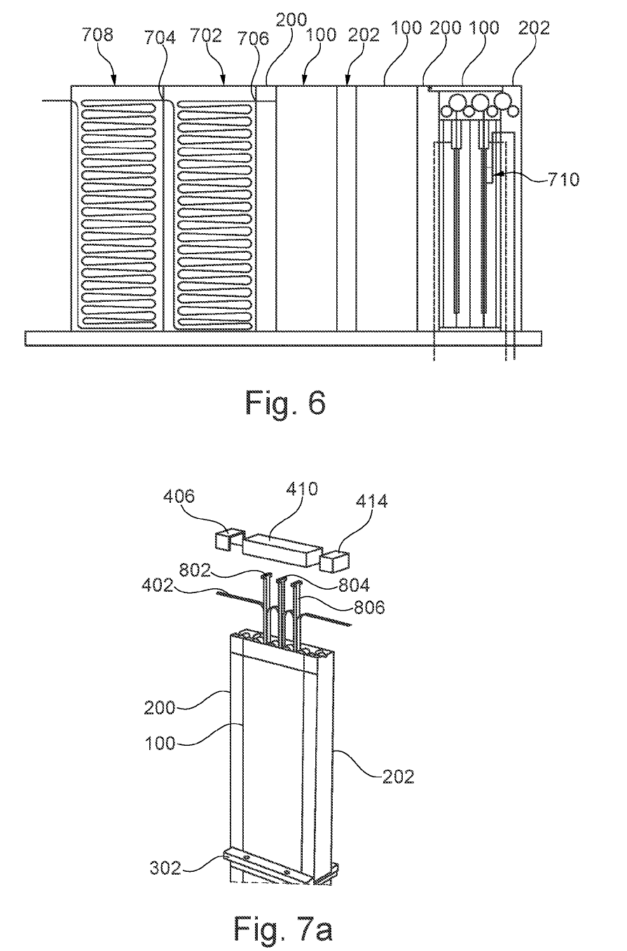

FIG. 6 illustrates a system including tape delivery cartridges;

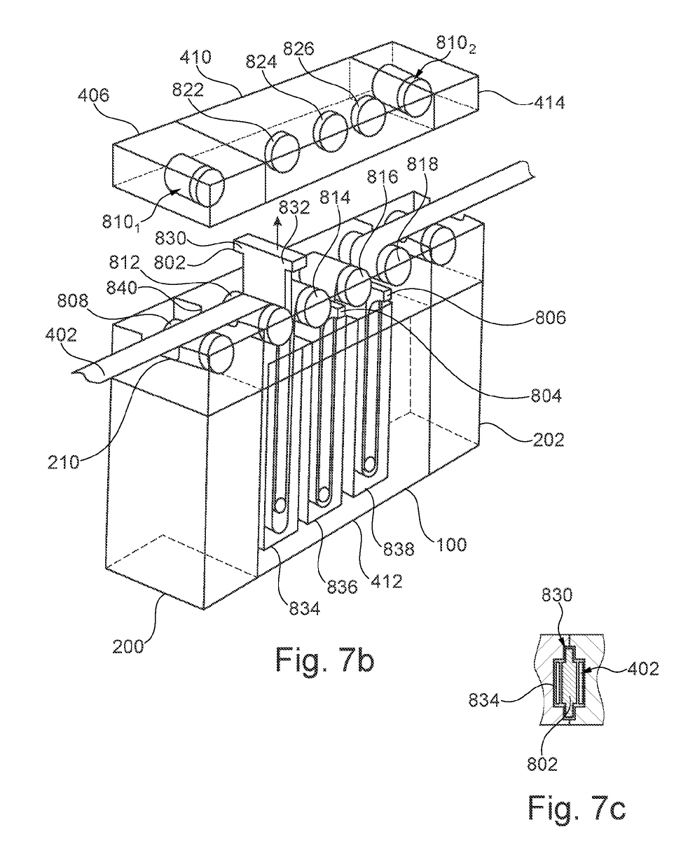

FIGS. 7a and 7b illustrate another system for contacting a solid phase with a fluid phase;

FIG. 7c illustrates a top view of a projection of FIGS. 7a and 7b;

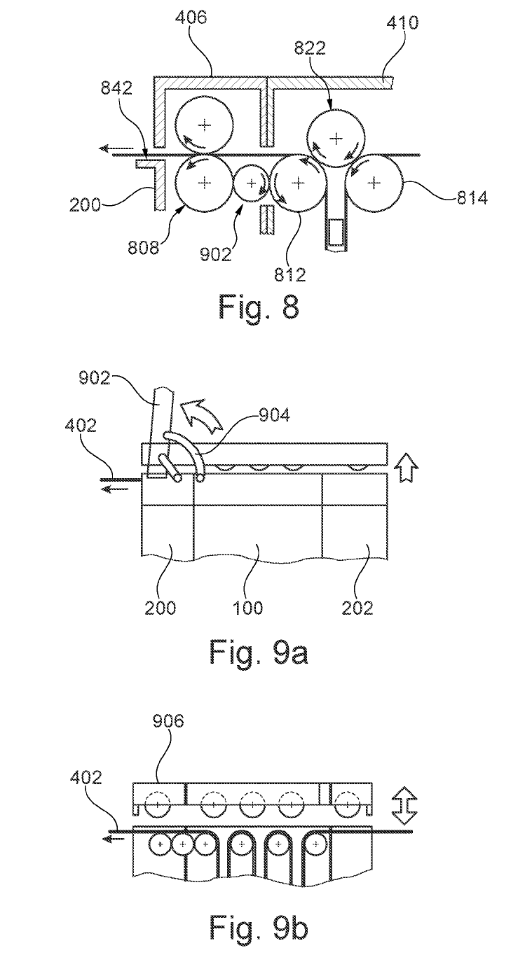

FIG. 8 illustrates a part of a system including another arrangement of a drive mechanism;

FIGS. 9a and 9b illustrate different lid arrangements;

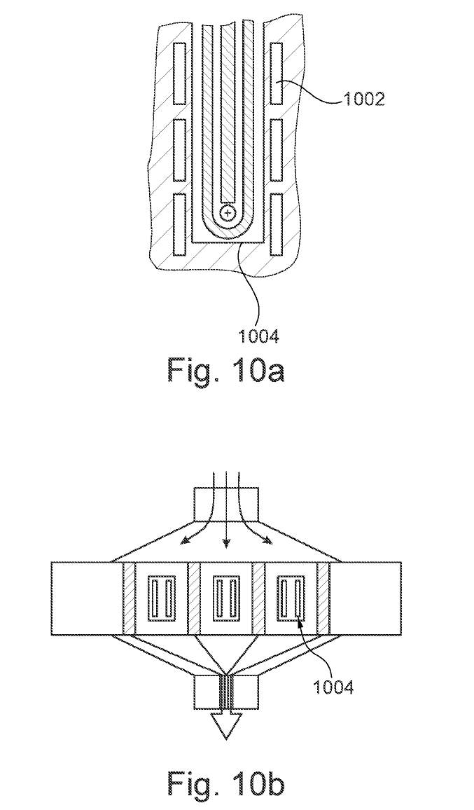

FIGS. 10a and 10b respectively illustrate a side view and top view of a cooling system for a reaction module;

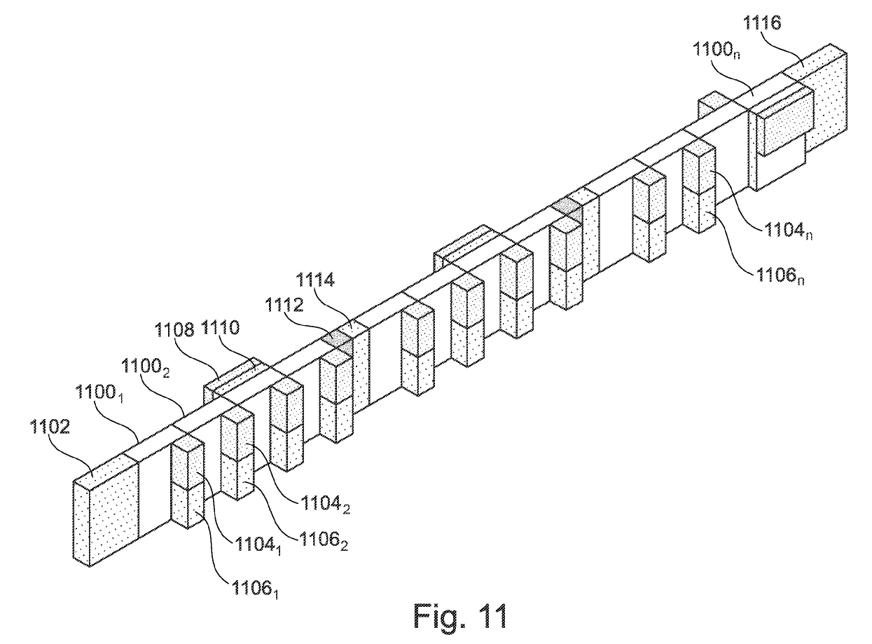

FIG. 11 illustrates another system for contacting a solid phase with a fluid phase;

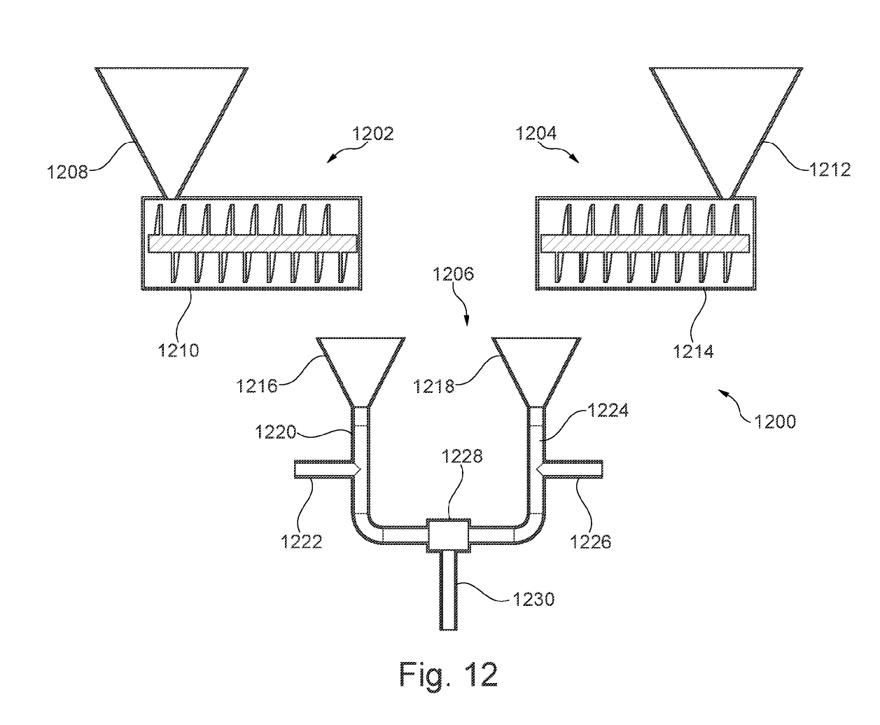

FIG. 12 illustrates a reagent activation module;

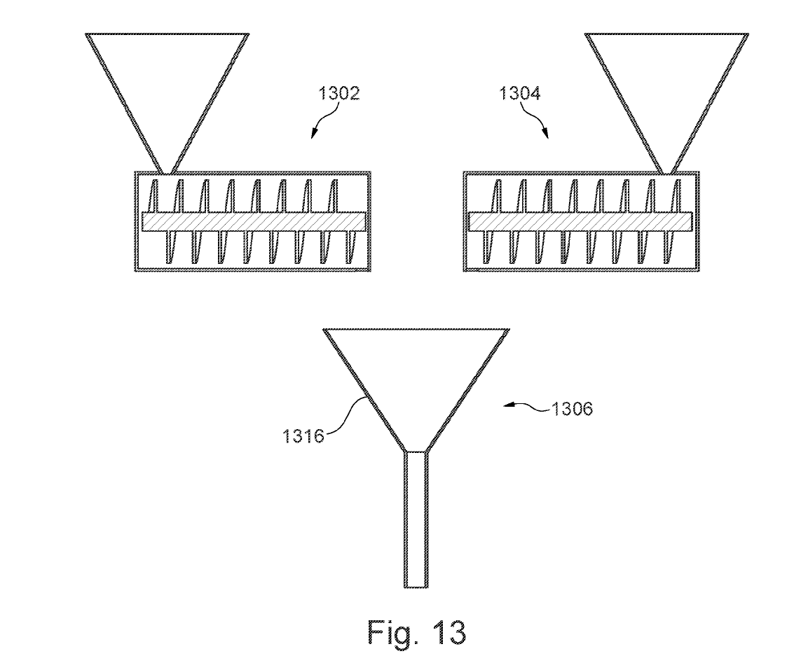

FIG. 13 illustrates another reagent activation module; and

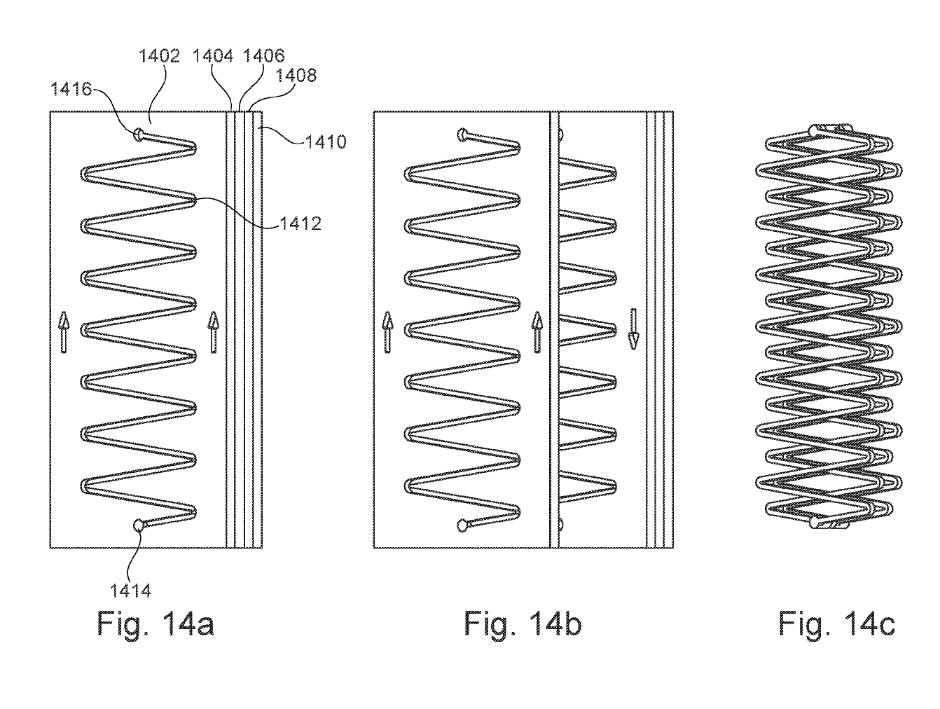

FIGS. 14a, 14b and 14c illustrate an incubation module.

### DETAILED DESCRIPTION

Included in the invention are apparatuses and methods for use in solid phase synthesis. Solid phase chemistry will require no elucidation for the skilled reader but, nonetheless, the reader is directed to the publications mentioned under the heading “Background” for further information on materials and methods which may be used in solid phase synthesis.

The invention generally relates to any process which involves contacting a mobile solid phase with a mobile fluid phase. During the process, therefore, the solid phase moves or is able to move; for example the movement of the solid phase may be a movement which would for practical purposes be considered continuous (including continuous movement driven by a stepper motor, which in fact rotates in high frequency steps). In some embodiments, the solid phase is stationary during performance of a process and then moved on to another apparatus to be subjected to another process. In other embodiments, the solid phase moves intermittently during performance of a stage of a process. The fluid phase flows during at least part of a process of the disclosure and it may flow continuously. Thus, the invention includes embodiments in which the solid phase is contacted with, e.g. surrounded by, a stream of fluid during part or all of a process. A fluid may flow continuously during a process but in some embodiments fluid flow is discontinuous. In many embodiments, both the solid phase and the fluid phase move continuously between the beginning and the end of a process.

The fluid phase may be a liquid. As will become apparent, a fluid is typically a liquid but may be a gas. The liquid may be a flowable foam or a flowable gel. Alternatively the fluid may be a gas. For the purpose of this disclosure, the term “liquid” includes liquid-like materials, for example foams or gels.

FIGS. 1a and 1b show a system 300 for contacting an elongate solid phase with a fluid phase. The system includes a reaction module 100, a first service module 200 and a second service module 202.

As used herein, a “reaction module” is an apparatus for performing an action on a solid phase. For example, a reaction or a washing stage may take place. When a solid phase is contacted with a fluid phase a reaction may take place to chemically change the solid phase, or the fluid phase may be used to wash components from the solid phase.

As used herein, a “service module” is an apparatus for supplying or receiving a fluid supply to or from the reaction module, and optionally supplying or receiving a solid phase to or from the reaction module. Supplying or receiving a solid phase may also include allowing a solid phase to pass through the service module so as to reach the reaction module or to exit the reaction module. The service module may optionally supply or contain further elements used for servicing the reaction process in the reaction module (described in more detail later).

The general concept of the system is to recreate a multi-step synthesis process on a solid phase material. This process usually includes one or more steps of reacting, cleaning, and analysing, which may be performed any number of times on the solid phase.

Reaction modules may be connected in series, separated by service modules, so as to form a plurality of reaction zones and/or rinsing zones. The reactions and rinses may be the same or different in the different zones.

While the solid phase is a ‘constant’ that will pass through plural zones (and plural reaction and service modules), the fluid is likely to be different for each zone (reaction module) via inlets/outlets in the service modules.

In FIG. 1b, the first service module 200 is operably connected to a side of the reaction module 100. The second service module 202 is operably connected to a further side of the reaction module 100. In this way both fluid and solid phases can be supplied to the reaction module 100 and retrieved from the reaction module 100.

The reaction module 100 includes a conduit for the passage of the fluid phase and the solid phase. In the conduit the solid phase is contacted with the fluid phase, and thereby the reaction or washing or other action takes place. The conduit includes a fluid input port 104 and a fluid outlet port (not visible), respectively connected to the first service module 200 and second service module 202. The reaction module 100 is configured for passing an elongate solid phase between the first service module and further service module via the conduit. In this example the reaction module 100 is configured to include a solid phase input port (not visible) and solid phase output port 106.

As can be seen in FIG. 1a, the second service module 202 includes a fluid port 208 that will connect and seal with the fluid outlet port of the reaction module 100. The second service module 202 also includes a solid phase port 212. The modules and ports are provided so as to releasably connect the ports to corresponding ports on the adjacent module.

The solid phase is aptly elongate, e.g. a ribbon, tape, web or cord. The elongate solid phase may optionally be a ribbon having a width of between 10 mm and 50 mm for example, more aptly around 25 mm.

The elongate solid phase may comprise a substrate, for example a natural or synthetic polymer, of which cotton and other cellulosic materials are an example. Alternative materials are described later in this specification as well as previously herein under the heading “Background”.

Alternatively the solid phase may comprise a plurality of solid particles, e.g. beads. The solid particles may be a polyethylene glycol or a copolymer, for example. The solid particles may be TentaGel™ beads, which is a PE glycol resin. The TentaGel™ beads may have a diameter of from 60 to 180 μm, for example from 60 to 100 μm or from 150 to 180 μm. The beads will typically be contained within a porous material. The porous material may be in the form of a tube in which the beads are contained. The pore size of the porous material will typically be less than the size of the beads. Thus, the pore size of the porous material may be less than 150 μm, less than 100 μm, less than 50 μm or less than 25 μm. The porous material may be a woven fabric (e.g. polypropylene mesh) or it may be a polymeric membrane.

FIG. 2 shows the reaction module 100 and first and second service modules 200,202. FIG. 2 also illustrates a port 210 in the service module 200 through which the elongate solid phase may be fed. For example the solid phase may be moved from another service module or reaction module (not shown), through the service module 200, through the reaction module 100, and through the service module 202.

FIGS. 2 and 3 also illustrate an optional holding element, in this case a base rail 302. The holding element acts as a convenient frame to attach one or more reaction modules and/or service modules.

The base rail 302 acts as a holder, to which reaction modules and service modules can be added or connected to. The base rail has a tray 304 that corresponds in size to the depth dimensions of the reaction modules and service modules. In this way one or more modules can be added in series to the base rail in a convenient manner.

In this example the sides of the reaction module and sides of the service modules are each mating faces.

FIG. 3 shows an example in which the modules are fitted, in series, in the order of: service module 200-reaction module 100-service module 202-service module 204-reaction module 102-service module 206. That is, each reaction module has ‘its own’ set of two service modules, one prior to and one after the reaction module in the series. The two service modules that sandwich a reaction module between them act to deliver and receive the solid phase, and deliver and drain a fluid phase, for the reaction module. It will be appreciated that the solid and fluid phases could be both delivered from a first service module and both received by a second service module, or the solid phase could be delivered by a first service module, and the fluid phase could be delivered by a second service module, i.e. flowing in the opposite direction to the solid phase. Aptly, the fluid phase does flow in the opposite direction to the solid phase.

It will also be appreciated that alternatively, the reaction modules and service modules could be provided alternately, i.e. in the order: service module-reaction module-service module-reaction module, etc. In this case, a (single) service module is capable of performing the necessary functions to service a reaction module immediately before it in the series (e.g. passing through a solid phase and draining fluid phase away), and servicing a reaction module immediately after it in the series (e.g. passing through the solid phase and providing a fluid phase to that reaction module).

Thus, the modules may be connected in a repeating pattern.

In addition, modules may be fitted with multiple reaction modules situated between a set of service modules, i.e. in the order: service module-reaction module-reaction module-service module. In this manner the reaction time, whereby the solid phase is in contact with the fluid phase, can be increased.

Thus, the length of a reaction zone, various reaction zones, or the entire passage for the solid phase to pass through may be selected in accordance with the reaction time required.

FIG. 4a shows a cross section through an assembly, which could be the assembly 300 of FIG. 1b, having a reaction module 100, a first service module 200 and a second service module 202. FIGS. 4b and 4c show a cross section through another assembly, having a reaction module 500, a first service module 200 and a second service module 202, which would also look like the assembly 300 in perspective view.

The assembly of FIG. 4a has a reaction module 100, a first service module 200 and a second service module 202. A side 214 of the first service module 200 is operably connected to a side 108 of the reaction module 100. A side 216 of the second service module 202 is operably connected to a further side 110 of the reaction module 100. In this case the sides are mated and sealed. It will be appreciated that the service modules 200,202 can be configured such that further sides (e.g. side 218 of the first service module, which is opposite the side 214, and side 220 of the second service module, which is opposite the side 216) are connectable to further reaction modules or service modules.

The reaction module 100 includes a conduit 112 for the passage of the fluid phase and the solid phase. In the conduit 112 the solid phase is contacted with the fluid phase, and thereby a reaction or washing or other action takes place. The conduit extends from a side 108 to a further side 110 of the reaction module.

The conduit 112 of the reaction module includes a fluid input port 104 and a fluid outlet port, respectively connected to the first service module 200 and second service module 202. The reaction module 100 is configured for passing an elongate solid phase between the first service module and further service module via the conduit. In this example the reaction module 100 is configured by including a solid phase input port and solid phase output port 106.

The first service module 200 includes a conduit 222 for connecting with the conduit 112 of the reaction module 100. The conduit 222 extends from the side of the service module 214 where a fluid port of the conduit 222 mates with a fluid port of the conduit 112, to a further side of the service module, which is the lower side 226. Although they have been termed fluid ports, the fluid ports described herein may be a simple interface, i.e. an end of a conduit that meets an end of another conduit to form a continuous passageway. The conduit 222 of the service module is aptly the same or similar cross section dimensions to the conduit 112 of the reaction module.

Similarly to (and as a mirror image of) the first service module 200, the second service module includes a conduit 224 for connecting with the conduit 112 of the reaction module 100.

Thus, the fluid ports are effectively an interface or an end of the fluid conduits; ends of conduit 112 (represented by dotted lines in FIG. 4a) are respectively connected to the conduits of the adjacent service modules.

Similarly, the solid phase ports are an interface between two modules for allowing the solid phase to pass. In this case the interface may be respective holes in the side of the modules that correspond in position, to allow a solid phase to pass therethrough.

As can also be seen in FIG. 1 a, the second service module 202 includes a fluid port 208 that will connect (and seal) with the fluid port of the reaction module 100.

The second service module 202 also includes a solid phase port 212. The modules and ports are provided so as to releasably connect the ports to corresponding ports on the adjacent module.

In this example, the solid phase is a ribbon 402. In use, the ribbon 402 is fed into the first service module 200 at arrow A. The ribbon 402 enters the solid phase input port of the first service module. The ribbon is fed through the first service module and through the solid phase output port of the first service module and solid phase input port of the reaction module. Then ribbon continues through the solid phase output port of the reaction module and solid phase input port of the second service module. The ribbon continues out of the solid phase output port of the second service module at arrow B.

Each module thus includes an input and output port for the ribbon and a passageway therethrough for the ribbon to pass through or lay.

The ribbon may be fed through the apparatus with a pulling action and/or a pushing action, for example by use of suitable rollers, gears and motors (an example will be described later with respect to FIG. 4c).

Fluid is fed from arrow C through a fluid phase entry port of the second service module, into the conduit 224, and through a fluid phase outlet port of the second service module. The fluid enters a fluid input port of the reaction module, into the conduit 112, and through a fluid outlet port of the reaction module. The fluid enters a fluid input port of the first service module, into the conduit 222, and through a fluid outlet port of the first service module at arrow D.

The fluid phase may be fed into the conduits by a pump or other suitable mechanism.

The conduit 112 includes a chamber 404 having an open area on its upper side. The open chamber allows the ribbon 402 to be fed into and out of the chamber. This arrangement enables the solid phase to contact the fluid phase.

The conduit may aptly provide a small clearance (e.g. between 1 mm and 5 mm) beyond the width of the ribbon.

In this example, each of the first service module, reaction module and second service module is provided in two portions, with a lid portion and a body portion. The first service module 200 includes a lid portion 406 and a body portion 408. The reaction module 100 includes a lid portion 410 and a body portion 412. The second service module 202 includes a lid portion 414 and a body portion 416. Each lid portion is releasably connected to a respective body portion.

The lid portion 410 of the reaction module 100 has a projection 418. The projection extends from the lid portion 410 towards and into the body portion 412. The projection is provided on an underside of the lid portion, such that when the lid portion is closed, the projection extends into the chamber 404. In this case the projection is coupled to the lid portion by adhesion. Alternatively the projection could be coupled to the lid portion in other ways, e.g. by fastening, connecting or being formed as a continuation (of a continuous material) with the lid portion. Alternatively, the projection could be a separate piece to the body and lid portions, which sits or hangs within the body portion, e.g. connecting at an upper area of the body portion and hanging down into the chamber (e.g. as described later in relation to FIG. 7a).

The projection has a height dimension that is substantially, but slightly less than, the height of the chamber. Thus there remains a lower portion of the chamber that remains open for the fluid and solid phases to pass. Also, the width of the projection is smaller than the width of the chamber (in the cross sectional view as shown in FIG. 4a). Thus there remains width portions of the chamber that are open for the fluid and solid phases to pass. The projection has a depth measurement that is substantially equal to the depth of the chamber. The depth measurement of the projection is such that the projection fits into the chamber and abuts the walls of the chamber.

In other words, the presence of the projection 418 within the chamber 404 acts as an internal wall that creates a flow path for fluid therearound. That is, the flowpath is an approximate U-shaped path (in the cross section shown in FIG. 4a) in which fluid can flow down, across and then upwards (but generally not around the sides of the projection in the depth direction that abuts the chamber wall).

In the same pathway, the solid phase (ribbon) 402 will also pass. The pathway creates an environment (or ‘reaction zone’) for the solid and fluid phase to contact each other.

In this embodiment the ribbon 402 moves from left to right, while the fluid flows from right to left. Thus the ribbon flows against the fluid current. A predetermined reaction may occur in the pathway.

FIG. 4b shows an arrangement similar to that in FIG. 4a. However, the reaction module 500 is modified compared to the reaction module 100. That is, the reaction module 500 includes a conduit 506 that has two chambers 502,504. Correspondingly, there are also two projections 508,510 that lie within the respective chamber. The remaining features of the apparatus are the same as the example of FIG. 4a and will not be described again. It will be appreciated that the two chambers increases the length of the ‘reaction zone’ for the solid phase to contact the fluid phase, and thus the time length for any reaction can be increased (when comparing fluid phase movement and solid phase movement fed at the same rates). One, two, three, or more chambers in the conduit of the reaction module may be chosen to suit the required use.

FIG. 4b is an example where the solid phase can move in the same direction as the fluid phase. Alternatively the fluid and solid phases can move in opposite directions.

FIG. 4c shows an arrangement similar to that of FIG. 4b. However, some optional additional details are shown in FIG. 4c.

The arrangement of FIG. 4c includes various rollers to guide the movement of the solid phase through the system. The arrangement includes a roller 512 in the first service module 200, rollers 514 in the reaction module 500, rollers 516 in the chambers, and rollers 518 in the second service module 202. Optionally at least one of the rollers is a driven roller. In the arrangement shown roller 518 is connected to and driven by a motor 520. Options for the drive mechanism to drive the roller(s) include individual motors, gear drives, worm drives, o-ring belt drives and electromagnetic drives.

The reaction module 500 of this arrangement also includes an outer containment body 522, and two inner reaction units 524 for the chambers 502,504. The outer containment body 522 may be of stainless steel, for example. The two inner reaction units 524 may be of PTFE, for example. Moreover, in the same manner as the arrangement in FIG. 4a, the reaction module has a lid portion 410 that seals with the reaction module. This seal or any other seal may be of DuPont™ Kalrez® perfluoroelastomer parts (FFKM), for example, which is a high performance elastomer part. The lid portion is connected to the reaction module.

There is provided a dual containment system for the reaction chamber. The wall of the inner reaction unit 524 acts as a first barrier to fluid. The wall of the outer containment body 522 acts as a further barrier to fluid. This will help ensure no fluid is leaked from the system. Thus the operator(s) and the environment are protected in a case where the primary chemical containment system fails. Optionally the closing of the lid portion may further act to help seal the fluid within the reaction module.

Again in the same manner as the arrangement in FIG. 4a, the first service module 200 includes a lid portion 406 and the second service module 202 includes a lid portion 414. Each lid portion is releasably connected to a respective body portion. In the arrangement shown, the reconnection of the lid portion 414 serves to engage the motor 520 and the driven roller 518 with the ribbon. Rotation of the driven roller 518 about its central axis will result in the ribbon being guided in the direction shown, i.e. the ribbon is pulled through from the first service module, reaction module, then to the second service module and onwards.

The arrangement of FIG. 4c also shows a number of o-ring seals 526. These o-ring seals help to prevent leakage of fluid from the various conduits, particularly at the interface between adjoining modules, where conduits are joined at inlet and outlet ports, and between inner and outer containment bodies. A person skilled in the art will be able to identify the required position of seals such that fluid does not leak from conduits or the interfaces between conduits. Moreover, the person skilled in the art will be able to recognise that different types of seal or methods of sealing the fluid within the conduit may be used.

In the arrangement of FIG. 4c, there is also shown a rubber connecting hose 528. The connecting hose connects the conduit 222 of the first service module 200 to a further conduit 530 located in both the first service module and reaction module. The conduit 530 has an elbow bend and connects between the connecting hose 528 and the chamber 502. As such, the conduit 222 connects with the conduit 528, which in turn connects with the chamber 502. The second service module 202 has the same conduit features as the first service module.

As shown in FIGS. 5a and 5b, rather than a tray or base rail (as per FIG. 2), the arrangement of service modules and reaction modules are arranged in-line, and (each) adjacent a service ducting module 604. The service ducting module 604 is an elongate housing that extends at least as long as an arrangement including a maximum number of service modules and reaction modules required. The service ducting module 604 provides services e.g. fluid supply, fluid drain and/or electricity to be connected with the service modules.

The first service module 200 of the apparatus may have a number of ports 602 respectively in communication with the interior of conduits 222,612,614 to allow a fluid phase, or a service (e.g. an electrical connection), to enter a conduit, flow through it and exit it.

FIG. 5b shows the service ducting module 604 containing a plurality of conduits 606,608,610, which connect to (and seal with) the conduits 222,612,614. The plurality of conduits 606,608,610 will also connect (not shown) to other conduits of other service modules being used. Here the connections are made using a push-fit seal.

In the arrangement shown there are three conduits within the service ducting module 604. A fluid phase (liquid or gas) is delivered through conduit 608, and a power supply is delivered through conduit 610. The conduit 606 is for fluid waste, i.e. fluid that has been used in a reaction module and is taken away via this conduit 606.

In the first service module 200, the conduit 222 connects the fluid phase (liquid or gas) from the conduit 608 in the service ducting module to the chamber of the reaction module 100. That is, the liquid or gas to be reacted with the solid phase is delivered via the conduit 222 to the reaction zone. The conduit 614 connects the power supply from conduit 610 to a drive roller 616 for driving gears (e.g. gear 618) to move the ribbon 402 through the system of modules.

Each conduit in the service ducting module is connected externally to a source of its respective contents, for example the power supply conduit 610 is connected to an external power supply. There may be any number of conduits in the fluid phase delivery module. An optional further connection would be a signal conduit, e.g. for providing signal wiring for a sensing apparatus, for example a tension sensing apparatus. A further optional connection would be a gas supply conduit, e.g. for supplying argon or nitrogen gas to the reaction module and service module to maintain the solid phase (ribbon) in an inert atmosphere when not in the reaction zone.

The conduits of the service ducting module deliver their respective contents to the desired location, for example the power supply will be delivered to the drive system and the fluid phase will be delivered to the corresponding fluid inlet port.

In the arrangement shown the fluid phase ports 602 are towards the lower side of a side face of the first service module (in the position of use). Such a position would also allow the service ducting to be incorporated into the base rail 302 (i.e. incorporating the necessary conduits into the body of the base rail). However it will be appreciated that the ports 602 may be positioned on any exposed external face of the service modules (i.e. that does not mate with neighbouring modules). Thus, in an alternative to the arrangement of FIGS. 5a and 5b, rather than a service ducting module, the services can be supplied to the service modules via similar conduits and appropriate connections within a base rail that the service modules and reaction modules can sit in.

As shown in FIG. 6, there may be further provided a solid phase delivery cartridge 702 (in this case a ribbon delivery cartridge), that releasably connects with a service module, a reaction module, and/or another cartridge. Aptly the height, width and/or depth dimensions of the solid phase delivery cartridge are substantially equal to the corresponding dimensions of the service module or reaction module against which it can be located. The solid phase delivery cartridge provides a ready method of supplying ribbon in a format in which a sealed container houses a length of elongate ribbon folded in a serpentine manner, such that both the leading and trailing ends of the length are presented respectively at an exit port 706 and entry port 704 of the cartridge. Thus the length of ribbon can be connected to another length of ribbon provided in a next, similar cartridge 708, reaction module or service module.

In addition to the features described above, a system may be included whereby specific reaction modifying devices, reaction promoting devices (e.g. energy sources), reaction testing devices or reaction diagnostic devices (e.g. process analytics for checking the system is working as it should be), can be readily incorporated (bolted on), e.g. microwave, ultraviolet, heating (e.g. using infrared), cooling, ultrasound waves or other acoustic waves. In general these devices may be provided within a service module.

FIG. 6 includes an optional UV emitting device 710 that is provided for providing a particular UV wavelength of radiation towards the reaction zone in the reaction module. The UV emitting device is included for testing the fluid that is exiting the reaction module.

In this case the UV is used as a measuring tool. The device 710 is located in the reaction module itself.

Other reaction modifying/promoting/testing/diagnostic devices will be described later, in relation to FIGS. 11-14.

The arrangement shown in FIGS. 7a, 7b and 7c illustrates a system similar to that of FIG. 4a, but the projection is a separate piece that sits within the body portion of the reaction module. In this case there are 3 projections 802, 804 and 806. The projections sit at an upper area of the body portion 412 of the reaction module 100 and hang down into the respective chambers 834,836,838. Also, the lid portions 406,410,414 of the first service module, reaction module and second service module, are completely removeable from the body portions of the respective first service module, reaction module and second service module.

More specifically, the projection 802 is formed with an approximate T-shaped cross section (in the depth direction of the apparatus). The projection 802 thus has two arms 830,832 at an upper end of the projection. The arms sit upon a shelf created by the upper edges of the chamber 834. A plan view of the projection 802 sitting in place is shown in FIG. 7c. The projection 802 is therefore insertable into, and removeable from, the chamber 834 (e.g. as indicated by the arrow).

The projection 802 has similar relative dimensions to the projection 418, i.e. dimensions that allow a pathway to be formed in the chamber around the projection (e.g. a U-shaped pathway). Insertion of the projection into the chamber forms a pathway suitable for the fluid phase and the solid phase to flow through.

A method of threading a ribbon through the system shown in FIGS. 7a, b and c will now be described. Referring to FIG. 7a, the arrangement has a first service module 200, reaction module 100 and second service module 202 (which could be those shown in FIG. 1a). In this method a first step involves the removal of the lid portions 406,410,414 of the first service module 200, the reaction module 100 and the second service module 202. As shown in FIG. 7b, by doing so any parts of the drive mechanism that are housed within the lid portions will also be removed, which in this case includes rollers 810_{1,2} (pinch roller and drive gear) in the lid portion of the first and second service modules, and rollers 822, 824, 826 in the lid portion of the reaction module. The projections 802,804,806 are then removed vertically from within the chamber of the reaction module 100 according the arrow shown in FIG. 7b. Although this example describes an initial step for removing the lid portions and a further step for detaching the projections, the method is equally applicable (with a single step), removing both the lid portions and projections simultaneously, e.g. where the projections are adhered to, or part of, the lid portions.

Then, the ribbon 402 is laid over the first service module 200, reaction module and second service module (or the full set of modules used in a particular treatment or experiment). In the embodiment shown the solid inlet port 210 is no longer fully enclosed as a result of the removal of the lid portions. In the first service module, the ribbon is placed over the roller 808 in the body portion, and over solid phase entry port 210 and exit port 840. Likewise, in the body portion of the reaction module and second service module, the ribbon is placed over the rollers and solid phase entry/exit ports.

Thus a continuous length of ribbon passes over all the modules shown in the system. When the projections are replaced into their positions in the respective chambers 834,836,838, the ribbon is pushed downwardly into each chamber by the corresponding projection.

In this arrangement, replacing the lid portions will engage the rollers housed in the lid portions with the rollers situated within the corresponding body portion, and in doing so will reconnect the drive mechanism. When the lid portion of the first service module 406 is replaced onto the body portion of the first service module 200, the roller within the lid portion 810₁ engages with a roller 808 in the body portion of the first service module, such that the roller in the lid portion is located directly above the roller in the service module and the ribbon is located between the two rollers. The result is the same when the lid portion 414 of the second service module is replaced onto the body portion of the second service module 202.

When the lid portion 410 of the reaction module is reconnected to the body portion of the reaction module 100, the three rollers 822, 824, 826 housed within the lid portion 410 engage with the four rollers 812, 814, 816, 818 in the body portion of the reaction module, such that each of the rollers in the lid portion sits above and between two of the rollers in the body portion of the reaction module.

The system is then ready to be used for a treatment or experiment or such like. The solid phase and fluid phase(s) (and optionally other reaction variables) can be fed through the set of service module(s) and reaction module(s) as required.

Optionally, at least one of the rollers, located in any of the modules, may be driven such that the ribbon can be progressed through the apparatus at a controlled rate.

It will be appreciated by a person skilled in the art that there are many possible configurations for the rollers within the drive mechanism that allow the ribbon to be progressed through the modules and only one configuration is shown here. E.g. the roller (or plurality of rollers) in the lid portion of a service module may be offset from the corresponding roller (or plurality of rollers) in the service module as opposed to vertically above it.

In the embodiment shown, the replacement of the lid portion of the reaction module further serves to seal the assembly, bring rollers together, and set the ribbon in place.

As per the arrangement of FIG. 6, additional ribbon may be joined and added to the system from a cartridge, without requiring the lid portions to be opened. I.e. the end of a first piece of ribbon may be adhered to the beginning of a further piece of ribbon from a new cartridge. Thus the reaction taking place need not be halted.

FIG. 8 shows a further drivetrain arrangement, similar to that in FIG. 7b. In this arrangement there is an additional intermediate connecting gear 902 situated between the roller 808 in the first service module and the roller in the reaction module 100, such that rotational motion is directly transferred between rollers, assisting the movement of the ribbon through the system. An example of the possible direction of rotation of each roller is indicated by the arrows in the figure. FIG. 8 additional shows an option feature of a lip 842 extending from the solid phase output port in the service module. The lip extends outwardly so as to allow a ribbon to rest on the lip and also helps prevent any fluid phase from dripping externally of the system.

FIGS. 9a and 9b show different lid portion attachment arrangements.

FIG. 9a shows an arrangement in which a lid 902 is connected to the service module via a hinge arrangement 904. In this arrangement, various rollers may be housed in the lid portion, and the projections may be separate pieces, as described with respect to FIG. 7b for example. In this case the lid 902 is a unitary piece that covers a first service module, a reaction module and a second service module.

Of course the lid can be any size to cover one or more modules in accordance with the requirements of the system.

FIG. 9b shows a further arrangement, in which a lid portion 906 is a unitary piece that lifts directly from the body portions of the modules. The lid portion 906 may include handles or straps. In this case the lid 906 is a unitary piece that covers a first service module, a reaction module and a second service module. The projections (not shown) may be attached to or formed with the lid portion, or may be separate pieces.

The system may optionally include a cooling system for removing heat from the reaction module. FIG. 10a shows a chamber 1004 in a reaction module. There is shown a cross section of a plurality of cooling channels 1002 located in close proximity to the chamber 1004. A cooling fluid, which may be air or any other suitable gas or liquid, is fed through the cooling channels such that heat generated in the chamber from the desired reaction is transferred to the cooling fluid and is subsequently removed from the system.

FIG. 10b demonstrates the path the cooling fluid takes as directed by the fluid channels shown as a top view of the modular assembly. In the arrangement shown the cooling fluid travels into the reaction module at an inlet before being directed through the plurality of cooling avenues, whereby each cooling avenue directs the cooling fluid past one of the chambers, in a direction perpendicular to the direction of the modular production line. Each cooling avenue may include a plurality of cooling channels as shown in FIG. 10a. The cooling fluid may be transported from a service module or from another supply. The cooling fluid may be supplied from any direction.

It should be appreciated that the cooling channels may direct the cooling fluid in any suitable direction provided it allows for heat removal from the system and moreover there may be a plurality of ports for the cooling channel to enter or exit each reaction module.

Alternatively, but in a similar arrangement to that shown in FIGS. 10a and 10b, the system may include a heating system for adding heat to the reaction module. A heating fluid may be pumped into one or more heating channels near the chamber.

The present invention also relates to a method, e.g. a method of solid state synthesis, which uses a system of the first aspect. The process typically comprises:
passing the mobile solid phase from the first service module to a further reaction module or service module, via the conduit of the reaction module; and
causing a fluid phase to enter the conduit of the reaction module through a first input port, flow through the conduit and leave through a first fluid output port.

The method may be a single step of a solid phase synthesis, e.g. the solid phase synthesis of a polymer.

The method may be the solid state synthesis of a polymer, in which case the method typically comprises:
passing the mobile solid phase through a series of the conduits of the reaction modules;
causing a series of independently selected fluid phases to enter the conduits of the reaction modules through the respective fluid input ports, flow through the conduits and leave through the respective fluid output ports to provide the desired polymer attached to the substrate; and
cleaving the polymer from the substrate to provide the desired polymer.

The cleavage step may be performed in a system of the first aspect or it may be performed in a separate reaction vessel.

The polymer may be a polypeptide. The polymer may be a polynucleotide.

The elongate solid phase may comprise a species selected from a polymer and one or more synthetic building blocks for a polymer attached to a substrate; the polymer may be a biological polymer or a non-biological polymer.

The process may serve to modify the substrate, for example activate it, functionalise it or change its functionality, in any of those cases typically to prepare the solid phase for attaching a substance to it. Thus, the solid phase may have a substance attached to it, often covalently but sometimes non-covalently. Non-covalent attachment may be adsorption; it may involve hydrogen bonding, ionic bonding or van der Waals forces, or a combination thereof. A substance may be a spacer or a linker, or a combination thereof, whose function is to enable or facilitate tethering, e.g. covalent bonding, of a second substance to the solid phase. A substance attached to a substrate may comprise a starting material or an intermediate in a synthetic process, for example a monomer, oligomer or intermediate polymer formed as an intermediate in the preparation of an end product polymer. An end product may therefore comprise a substance having repeating units; the smallest such substance is a dimer, e.g. a dipeptide; more often the number of repeating units is greater than two and an end product may be a polymer, e.g. a biological polymer or a non-biological polymer. A substance having repeating units may be a poly(amino acid). As biological polymers may be mentioned polypeptides, polynucleotides and polysaccharides. As non-biological polymers may be mentioned organic semiconductor polymers.

Particular polymers which may be made are organic semiconductor polymers, for example made following the procedure of Turner et al (see above). A suitable substrate is functionalized with hydroxy groups which are further functionalised to provide a reactive germyl linker (see Scheme 3 of Turner et al).

Included amongst synthetic processes are those in which a substance attached to a substrate is subjected to a process comprising synthetic modification. For example, a biological or non-biological molecule, e.g. biological oligomer or biological polymer, may be modified by attaching one or more saccharides, e.g. to provide an amino acid, polypeptide or lipid with an attached group comprising one or a plurality of saccharides. A molecule, e.g. completed polymer, for example a completed PEG or polysaccharide, may be coupled directly or indirectly to a substance (e.g. a polypeptide) attached to a substrate; one example of indirect coupling is through a poly(amino acid) which includes a sequence cleavable by a protease. Such a cleavable poly(amino acid) may be used to couple a lipid, for example a fatty acid, to another molecule, e.g. a polypeptide.

A substance attached to a substrate may be a starting material or intermediate in a synthetic process for forming a molecule not comprising repeating units, e.g. a small organic molecule having a molecular weight of, for example, less than 1000, optionally less than 500.

Where a solid phase is an intermediate or starting material for a synthesis attached to it, the solid phase is subjected to a solid phase synthesis process. In other processes, a substance attached to a solid phase is not intended for synthetic use but for another process, for example performance of an assay. Typically, an assay involves exposing a substance to a further substance and monitoring for, and optionally measuring, an interaction between the two; for example, the interaction may be binding or it may comprise a reaction or other change of state, as in the case of an enzyme and a substrate. An assay may involve exposing a substance to two or more further substances, for example to a first biological structure, e.g. molecule, and a second biological molecule which is able to interact with the first biological structure; in this case, the assay may involve monitoring for, and optionally measuring, any inhibition in the interaction caused by the substance attached to the substrate. A substance attached to a substrate for use in an assay may be a biological structure, e.g. biological molecule, or a synthetic molecule intended for use in an assay involving a biological structure or a structure having, at least in a qualitative sense, an activity of a biological structure, as in the case of a molecule comprising a fragment of a protein which fragment has, at least qualitatively, a binding activity of the protein.

The step of the solid state synthesis may be the addition of a monomeric component of the desired polymer by covalent bond formation to the substance attached to the solid phase. The step of the solid state synthesis may be the activation of the substance attached to the solid phase towards covalent bond formation, e.g. by forming an acid chloride or a mixed anhydride. The step of the solid state synthesis may be the removal of a protecting group from the substance attached to the solid phase. This is known as a deprotection step in peptide synthesis or a deblocking step in oligonucleotide synthesis. In these embodiments, the fluid phase will typically comprise a reagent suitable for effecting said transformation.

The step of the solid phase synthesis may be a washing step to remove unreacted reagents or by-products of the above described steps, e.g. by-products of protecting group removal.

The step of the solid phase synthesis may be the cleavage of the polymer from the solid phase to provide the desired polymer (e.g. protein or polynucleotide).

A typical solid phase synthesis may therefore comprise the following types of steps:

1. Pretreatment Steps

A synthetic process may comprise one or more initial pre-treatments before synthesis is commenced. A typical pretreatment serves to provide the solid phase with appropriate functional groups on which to start synthesis; as appropriate, spacers and/or linkers may be attached to the solid phase to facilitate attachment of synthetic building blocks. Unwanted functional groups may be capped to prevent reaction subsequently. Pretreatment may comprise an optional initial washing stage, followed by acid activation, attachment of a spacer, optional washing, capping, deprotection, optional washing and attachment of a linker, e.g. a Rink linker. Each of these stages may be carried out in a separate reaction module or a separate group of sequential reaction modules. A pretreatment step may involve use of an energy source.

2. Synthesis Steps

A solid phase synthesis will comprise at least two synthesis steps in which the solid phase is contacted with synthetic building block, e.g. a monomer or oligomer of a biological polymer or a future part of another organic molecule. As monomers may be mentioned amino acids, nucleotides and monosaccharides. As oligomers may be mentioned oligopeptides or oligonucleotides. The synthetic building blocks may comprise appropriate protecting groups (e.g. FMoc protecting groups). The synthetic building block is therefore contacted with the solid phase in a reaction module, and upon contacting the solid phase reacts with a reactive group to become bound to the substrate. A synthetic building block may be a first synthetic building block with which the solid phase is contacted, in which case it is coupled to the substrate either directly or via one or more intermediate moieties (e.g. a spacer and a linker). Alternatively, a synthetic building block may be a second or subsequent synthetic building block which reacts with a moiety already formed by one or more previous synthetic building blocks. A synthesis step may involve use of an energy source for example in order to accelerate the rate of the synthetic reaction.

3. A washing Step

Frequently, a solid phase is washed after being contacted with a reagent, in order to remove unreacted reagent. Such washing steps may occur after addition of a synthetic building block, after contacting a solid phase with a ligand or analyte, or after contacting the solid phase with some other reagent, for example an agent to add or remove a protecting group or to activate a functional group. The liquid phase (the washing liquid) may flow in the opposite direction to the direction of movement of the solid phase, i.e. the two phases may be in countercurrent. A washing step may involve use of an energy source for example in order to increase solubility of an unwanted substance or accelerate its dissolution.

4. A deprotection Zone

Solid phase synthesis may involve deprotection of a protected functional group of a moiety bound to the substrate prior to a synthesis step. Such deprotection is typically effected by contacting the solid phase with one or more reagents and, in such a case, a deprotection zone may contact the solid phase with a flowing fluid phase (usually a flowing liquid phase), for example using an apparatus of the invention, e.g. a stack of treatment devices. Deprotection may be effected or promoted by irradiation, for example with UV radiation. The fluid phase may flow in the opposite direction to the direction of movement of the solid phase, i.e. the two phases may be in countercurrent. A deprotection step may involve use of an energy source as previously described in order to accelerate the deprotection reaction.

A solid phase synthesis process may comprise the following steps in succession: a deprotection zone, a washing zone then a synthesis zone; in embodiments, the washing zone is dispensed with.

5. An activation Zone

Solid phase synthesis may involve activation of a functional group of a moiety bound to the substrate prior to a synthesis step. The fluid phase may flow in the opposite direction to the direction of movement of the solid phase, i.e. the two phases may be in countercurrent. An activation step may involve use of an energy source in order to accelerate the activation reaction. A solid phase synthesis process may comprise the following steps in succession: an activation zone, a washing zone then a synthesis zone; in some embodiments, the washing zone is not included.

The above list of possible steps is not exhaustive. For example, a synthesis may include a functional group transformation.

It is within the bounds of this invention that subsequent processes are performed on the polymer once it has been cleaved from the solid phase. Thus, it may be that the polymer is glycosylated or coupled with another biomolecule. It may be coupled with another biomolecule, e.g. a protein (where the polymer synthesised on the solid phase is a protein, it may be coupled with another protein) an oligonucleotide or an antibody. The method may thus comprise coupling the polymer with at least one species selected from: a polypeptide, a polynucleotide, a sugar, an oligosaccharide or an antibody. Said coupling step may be performed in a system of the first aspect but it is more preferably performed in a separate reaction vessel.

The polymer may be treated with an acid or a base to form a salt. Where the polymer is a protein comprising at least two cysteine residues, it may be that disulphide bridge is formed, e.g. by exposing the polymer to reducing conditions. It may be that the biopolymer is subjected to a global deprotection reaction, e.g. a deacetylation reaction.

FIG. 11 illustrates another embodiment of a system for contacting an elongate solid phase with a fluid phase. The system includes a solid phase delivery cartridge 1102, and reaction modules 1100₁₋ₙ, as described previously. The reaction modules are provided in series (so that a solid phase may pass from the cartridge, then through consecutive reaction modules).

In this embodiment, there is provided separate fluid service modules 1104₁₋ₙ and electrical service modules 1106₁₋ₙ. A set of a fluid service module 1104₁ and an electrical service module 1106₁ are used instead of a ‘combined’ service module that provides both fluid and electrical supply. That is, the fluid service module provides fluid services to the system, e.g. reagent supply (fluid phase supply), waste drainage (fluid phase removal), and/or a gas line (e.g. for inert atmosphere). The electrical service module provides electrical or electronic services to the system, e.g. electric power supply for the drive system, connections for sensors to a central control (e.g. flow rate, temperature).

It may be apt to separate the fluid services and electrical services for safety reasons or to simplify servicing, or to allow greater separation of the fluid and electrical connections on the reaction module.

In this arrangement the service modules are provided on a side of the system rather than being in series with the reaction modules. It will be apparent that the service modules need not be equipped for the solid phase to pass therethrough in this case. Rather, the solid phase may pass directly from reaction module to reaction module. The connecting interfaces for the services to be provided to the reaction modules may be positioned accordingly so as to enter/exit the reaction module at appropriate location.

This configuration may aptly allow a relatively simple arrangement of modules and relatively simple set up of the system with minimum operations, thereby speeding up assembly time.

There is also provided one or more reagent activation module 1108, discussed later with respect to FIGS. 12 to 14. There is also provided an incubation module 1110, analysis module 1112, drive module 1114 and a waste ribbon container 1116 at the end of the system.

The incubation module is similar to a reaction module in that it includes a channel of defined length through which the mixed coupling or activation reagents and monomers (e.g. amino acids) flow for a defined period of time. In this way, chemicals that have been mixed in the reagent activation module may be incubated for a predetermined time period. Thus, for example, if the monomer unit to be added is a N-protected aminoacid, the coupling reagents may be those used to convert acids to acid chlorides or anhydrides. The incubation module allows the desired reaction between the activating agent and the monomer to go to occur. In a further illustrative use of an incubation module, it may be advantageous to incubate the cleaved polymer in the cleavage solution for a period after being cleaved from the solid phase resin. In peptide synthesis, this may, for example, allow deprotection of some or all of the amine protecting groups from the peptide chain. This is likely to be the case where deprotection and cleavage occur under the same reaction conditions. The incubation module may thus serve to incubate the reagent stream or the product stream. The incubation module is thus provided at a location between the reagent activation module and a reaction module. Here it is on a side of the reaction module not mated with another reaction module.

There may optionally also be an analysis module 1112, including parts for analysing or detecting a parameter of the reaction stream, for example using a thermocouple, or UV spectroscopy, or infrared spectroscopy, or Raman spectroscopy, or nuclear magnetic resonance spectroscopy.

There may optionally also be a drive module 1114 that separately provides the necessary motors and gears etc. for moving the ribbon through the system. For example, the module may include a pair of squeeze rollers driven by a signal control of a central control system.

The drive module may include a tension measuring/monitoring element. For example, the tension element may include a wheel that would be free to move (e.g. vertically) in response to tension. The position of the wheel would be detected via sensor and then the speed of the drive motor adjusted to maintain the correct tension of the solid phase in the system.

The system may include a central control system for controlling various electrical components of the system. The central control system may be located anywhere that is connectable with all required electrical parts. The central control system may be connected with any or all of: the drive module, the electrical service module, the analysis module, the fluid moving system (e.g. pumps), various sensors, the movement of the feed of the powder input hopper, etc.

At the end of the series of modules there may be a waste ribbon container 1116 that receives the ribbon that has been through the system. The waste ribbon module is the final collection point for the ribbon after the necessary reactions, washes, etc. have been completed.

FIG. 12 shows a reagent mixing module (or reagent activation module). For example, as a starting material for the fluid phase, first and second reagents may be mixed together, e.g. amino acids and coupling agents, for use in the reaction zone, or amino acids and activating agents where an N-protected amino acid is activated before it enters the reaction zone. This may typically involve the provision of an amino acid (typically supplied in the form of a powder) dissolved in a solvent, and being mixed with the coupling agent or activation agent. It may be that the amino acid is mixed with a coupling agent or activating agent that is also supplied in the form of a powder, dissolved in another solvent. It is this embodiment that is depicted in FIG. 12 but it is within the scope of the invention that the coupling agent or activating agent is in the form of a liquid that is added into the reagent mixing or reagent activating module via an outlet.

Heretofore, the liquid provided to a reaction would generally be sourced from a supplier pre-mixed. The reagent activation module enables a liquid to be prepared at the appropriate time and ‘mixed to order’, avoiding settling and such like.

The reagent activation module 1200 includes two powder input hoppers 1202, 1204, and a mixing apparatus 1206. A first powder (e.g. amino acid) is provided to the input (funnel) 1208 of the first powder input hopper. A screw feed 1210 turns at a predetermined rate (e.g. under signal control of a central control system), and provides the first powder to the mixing apparatus at a set flow rate. Similarly, the second powder input hopper 1204 also has an input (funnel) 1212 and a screw feed 1214 that operate in the same manner as the first powder input hopper. So, a second powder (e.g. coupling agent) is provided to the second powder input hopper 1204 and exits at a set rate to the mixing apparatus.

The two powders each fall by gravity into a respective input (funnel) 1216, 1218 of the mixing apparatus.

The mixing apparatus 1206 thus has an input for receiving each powder. The input is a funnel in this case. The outlet of the first funnel 1216 is connected with a first tube 1220. A liquid supply tube 1222 connects with the first tube 1220. Liquid solvent (appropriate for dissolving the first powder) may be provided via the liquid supply tube 1222 into the first tube 1220. Similarly, the outlet of the second funnel 1218 is connected with a second tube 1224. A second liquid supply tube 1226 connects with the second tube 1224. Liquid solvent (appropriate for dissolving the second powder) may be provided via the liquid supply tube 1226 into the second tube 1224.

The first tube 1220 and second tube 1224 join at a T-piece 1228. The T-piece connects the two tubes and provides a single output tube 1230. Thus the two streams of solvated reagent are combined into a single stream in the output tube 1230. The stream of solvated reagents will likely require a period of time to react. Thus, the output tube may connect with an incubation module (e.g. module 1110 shown in FIG. 11 and described in more detail below in relation to FIG. 14).

In this way, the reagent activation module allows powdered reagents to be continuously dissolved in a solvent, and mixed with one or more further solvated reagents. The output tube 1230 may include mixing elements (not shown), e.g. baffles or stirrers if needed to combine the two solutions. The resulting solution may be incubated for the appropriate time, and go on to enter a reaction module in the main system.

FIG. 13 shows a variation on the reagent activation module 1200, in which two input powder supplies (hoppers 1302, 1304) are located to provide two powders to a single funnel 1316 of a mixing apparatus 1306.

A single powder material may be used for the system (from one or more supply), or two powder materials, or three or more powder materials. Aptly, in many reaction experiments, two powders will be needed, but this is not always the case. Any number of powder materials may be used.

FIGS. 14a, 14b and 14c show an example of how an incubation module may be arranged. The incubation module receives a solution from the reagent activation module, and is a conduit to the reaction module. The mixture from the reagent activation module spend a predetermined amount of time flowing through the incubation module so as to allow the desired reaction to occur, ready for introduction to the reaction module. Thus in a basic form, the incubation module could simply be a module including a tube of predefined length, through which the fluid may flow. In fact this may be all that is required, particularly for situations when the incubation module is a disposable feature.

FIG. 14a shows an arrangement with a series of plates 1402, 1404, 1406, 1408, 1410. The plates are relatively thin and flat sheets (having thickness or depth much smaller than their height and width). They may be formed from e.g. a polymer, for example PTFE. As can be seen, the plate 1402 has a channel 1412 that is provided on a side of the plate to form an open channel. It does not extend fully through the thickness of the plate. The other plates are of the same dimensions, and are provided with similar open channels (not visible in the view shown).

The start point 1414 of the channel is an open area that is connected with an input. For example, a flat plate of same dimensions (not shown) may overlay the plate 1402, and act to close the open channel 1412. A hole through that flat plate connects with the start point 1414. This forms a fluid input channel.

The end point 1416 of the channel 1412 is a hole that extends fully through the thickness of the plate 1402.

The plates may be arranged with holes at appropriate locations so that, upon stacking the plates together in a face to face relation, the start point and end point holes of the channels connect appropriately to create a single channel that extends consecutively through each plate. E.g. an end hole of a first plate meets a start hole of a next plate, and alternate holes are offset to avoid a hole extending right through all plates.

For example, as indicated in FIG. 14b, the channel may extend across the height direction of a first plate as shown by the upward arrows, through into a next plate, and down the height direction of the next plate (downward arrow), and so on. The length of the open channels may be designed to have a specific dwell time for a specific fluid moving speed, e.g. a 10 minute dwell time (with a particular pump speed used). Thus an appropriate number of plates may connected to suit a particular chemical reaction time.

FIG. 14c is a visual representation of the overall channel that will be formed by the stacking of the plates described above, without showing the plate surfaces. In this case the channel takes on a zig-zag formation, the zig-zag itself extending first in one direction e.g. upwardly, then in the opposite direction e.g. downwardly, then back in the opposite direction, etc.

Such an arrangement may provide an efficient use of space to create an extended length of incubation channel in a relatively small area.

Either the reagent activation module or the incubation module may also include a temperature control element for monitoring or control of the reaction occurring therein.

Various modifications to the detailed designs as described above are possible. For example, any number of reaction modules and service modules may be provided to suit the treatment to be performed. The service modules may provide any suitable service connections to service the reaction module(s). There are other ways of providing the solid phase to the system to those described above, e.g. a separate unit for pulling the solid phase provided at the front of the modules. If using a solid phase delivery cartridge, the ribbon may be housed in any suitable manner, e.g. serpentine folding, rolled up, etc. The chamber and projection in the reaction module may have any suitable shape and dimensions so as to allow a pathway to be formed to accommodate the fluid phase and solid phase entering and exiting the reaction module.

In some of the above described embodiments (e.g. FIGS. 5a and 5b), there is a single service ducting module to provide services to all the necessary service modules. Alternatively a service ducting module may provide services to a smaller set of service modules. For example one service ducting module may be provided for each set of say 3 or 4 services modules.

Although the above described embodiment of FIG. 1 may include reaction modules connected in series and separated by service modules, other arrangements are possible. The service modules may adjoin the reaction modules from any side, e.g. as per FIG. 11. As another example, a service module may be provided on the top side of a respective reaction module. Then reaction modules (with their respective service modules) may be connected together as needed. This may allow the service modules to be more easily removed from the system compared to some arrangements. Alternatively, a service module may be provided on the bottom side of a respective reaction module. Alternatively a service module may be provided on a vertical side of a respective reaction module. With these configurations, the solid phase may enter and leave a reaction module, and enter plural reaction modules in sequence, and service modules may provide the necessary services to/from each reaction module.

The reaction modules may be provided in an in-line configuration in its literal sense, i.e. in a straight line, or may be provided in a chain that changes direction.

The system may comprise any number of reaction modules. Typically the system may include 10 to 200 reaction modules, aptly 30 to 150 reaction modules, or 100 to 150 reaction modules.

With the above-described arrangement the supply and removal of fluid, and other peripheral services, are effectively detached from the apparatus where the reaction occurs (the reaction zone). In this way the reaction zone can be separated from the other necessary parts that are used in the reaction. With the other necessary parts separated into a specific unit as the service module, the whole system can be built up in units. The separate units can be designed to engage in series.

With separate modules that are built up, this avoids a more complex single system (as known in the prior art). A number of modules can be loaded together relatively easily to form a system. The tooling requirements and complexity of assembly and disassembly are reduced.

The modules can be connected simply into a dedicated production line, whereby the production line can be of any length with modules ordered in any combination provided each reaction module has a source of a fluid phase and a solid phase, e.g. from a service module, and an outlet to which fluid phase and solid phase are delivered. Each service module may carry out more than one of these tasks for a reaction module and may also carry out tasks for more than one reaction module by providing a connection to the appropriate conduit.

The modular arrangement and connection between the service modules and reaction modules allows for simultaneous connection of solid phase ribbon, liquid phase, mechanical drive trains and optionally other services.

Furthermore maintenance and cleaning of the apparatus is simplified in comparison to other systems, with the modular arrangement allowing the removal and treatment of single modules with minimal hindrance to the system as a whole.

The use of service modules provides the benefit that the number of separate components can be reduced, with the option of service modules servicing multiple reaction modules. The arrangement described allows for easy ribbon threading over the entire modular system without the requirement of hand-feeding the ribbon or dismantling a large number of components as in previous systems. Moreover the length of the ribbon can be increased by attaching additional lengths of ribbon contained within modules to the system to allow for a continuous feed to the production line. The connection of the additional ribbon may be automated wherein less intervention is required to provide a continuous supply of ribbon. In addition the arrangement allows for the length of each reaction channel to be varied to control residence time. This can be achieved either by changing the number of reaction channels in each reaction module and/or coupling multiple reaction modules together between service modules. Furthermore the apparatus allows for the means to produce specific reaction conditions to be bolted on, which could be (but are not limited to) microwave, ultra-violet, infra-red, ultrasound, other heating elements, thus providing greater flexibility and control over the length and conditions of reaction when compared to known systems.

The design of the modules allows for dual containment of the fluid phases provided for the reactions. This reduces the risk of fluid loss from the system and increases safety with regards to the use of hazardous fluids.

The arrangement described allows for the inclusion of a cooling system which can remove heat from the reaction module, or a heating system to add heat, or a combined and controlled heating/cooling system.

It will be clear to a person skilled in the art that features described in relation to any of the embodiments described above can be applicable interchangeably between the different embodiments. The embodiments described above are examples to illustrate various features of the invention.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A system for contacting a mobile solid phase with a flowing fluid phase comprising:
a first reaction module;
wherein the first reaction module comprises a conduit for the passage of a fluid phase and a solid phase, the conduit comprising a fluid input port and a fluid outlet port; and
a first service module operably connected to a first side of the first reaction module, the first service module for supplying and/or receiving the fluid phase to and/or from the first reaction module;
wherein the system is configured for passing a solid phase through the first reaction module, via the conduit.

2. The system as claimed in claim 1 further comprising a second reaction module, provided in series, such that the solid phase may pass through the consecutive reaction modules.

3. The system as claimed in claim 2 wherein the two reaction modules, and the first service module, are all configured to releasably connect to adjacent modules.

4. The system as claimed in claim 1, wherein the first side and a further side of the first reaction module are each a mating face; and
wherein the first service module has a mating face that is connectable with a respective mating face of the first reaction module.

5. The system as claimed in claim 1, wherein the conduit of the first reaction module comprises a solid phase input port and a solid phase output port;
wherein one of the solid phase input port and solid phase output port is provided in the first side of the first reaction module; and
wherein the other of the solid phase input port and solid phase output port is provided in a further side of the first reaction module.

6. The system as claimed in claim 1, wherein the first reaction module is releasably connected to the first service module.

7. The system as claimed in claim 2, wherein the fluid input port and fluid outlet port are provided on sides of each of the reaction modules so as to releasably connect and seal to corresponding ports on adjacent modules.

8. The system as claimed in claim 1 further comprising a holding element;
wherein the first reaction module and service module connectable to the holding element.

9. The system as claimed in claim 1 further comprising a service ducting module;
wherein the first reaction module and service module are connectable to the service ducting module; and
wherein the service ducting module is for providing services to the service module.

10. The system as claimed in claim 1 further comprising a solid phase delivery module configured to connect in series with the first service module or reaction module;
wherein the solid phase delivery module comprises a source of the solid phase.

11. The system as claimed in claim 1, wherein the first reaction module comprises a lid portion and a body portion; and
wherein the lid portion is removeable from the body portion so as to give access to the conduit of the first reaction module.

12. The system as claimed in claim 1, wherein the conduit comprises a chamber;
wherein the first reaction module comprises a projection that is at least partially insertable into the chamber; and
wherein the chamber and projection form a passageway as part of the conduit.

13. The system as claimed in claim 1 further comprising a drive train element for moving the solid phase through the system.

14. The system as claimed in claim 1, wherein the first service module comprises a fluid conduit for connecting with the fluid input port of the first reaction module.

15. The system as claimed in claim 1, wherein the first service module comprises a fluid phase supply or removal element; and
wherein the system further comprises a second service module comprising an electrical power supply element.

16. The system as claimed in claim 1 further comprising a reagent activation module for:
mixing and activating a reagent; and
providing the reagent to the first reaction module;
wherein the reagent activation module is provided on a side of the first reaction module, directly or indirectly.

17. A reaction module for contacting a mobile solid phase with a flowing fluid phase comprising:
a conduit for the passage of a fluid phase and a solid phase, the conduit comprising a fluid input port and a fluid outlet port;
wherein the fluid input port is provided in a first side of the reaction module, and the fluid outlet port is provided in a further side of the reaction module;
wherein the reaction module is configured to be operably connectable to a first service module, and a further service module or a further reaction module on the further side of the reaction module, for providing the fluid phase and the solid phase to the reaction module; and
wherein the reaction module is further configured for passing an elongate solid phase there through, via the conduit.

18. The reaction module as claimed in claim 17 further comprising a housing for housing the conduit;
wherein the first side and further side of the reaction module are respectively a side and further side of the housing.

19. The reaction module as claimed in claim 17, wherein the conduit further comprises a solid phase input port and a solid phase output port.

20. The reaction module as claimed in claim 19, wherein one of the fluid input port and fluid outlet port, and one of the solid phase input port and solid phase outlet port, are each provided in the first side of the reaction module; and
wherein the other of the fluid input port and fluid outlet port, and the other of the solid phase input port and solid phase output port, are each provided in the further side of the reaction module.

21. The reaction module as claimed in claim 17 further comprising a lid portion and a body portion;
wherein the lid portion is removeable from the body portion so as to allow access to the conduit.

22. The reaction module as claimed in claim 17 further comprising a projection;
wherein the conduit comprises a chamber;
wherein the projection is at least partially insertable into the chamber; and
wherein the chamber and projection form a passageway as part of the conduit.

23. The reaction module as claimed in claim 17 further comprising a drive train element for moving the solid phase through the reaction module.

24. The reaction module as claimed in claim 17, wherein the reaction module is configured to be releasably connectable to adjacent modules.

25. A service module for providing services to a reaction module for contacting a mobile solid phase with a flowing fluid phase, the service module comprising:
a housing having a side for operable connection with a reaction module;
the housing comprising a conduit for flowing fluid, the conduit having a first end for connection to a fluid supply or fluid drain, and a second end at the side of the housing for connection with a conduit in the reaction module;
wherein the service module is configured for providing one or more services to the reaction module.

26. The service module as claimed in claim 25 further comprising a driving element for moving an elongate solid phase through the service module, towards or from the reaction module.

27. The service module as claimed in claim 26 further comprising a motor to drive the driving element.

28. The service module as claimed in claim 25 further comprising a further conduit for flowing fluid, the further conduit having a first end for connection to a fluid supply or fluid drain, and a second end at a further side of the housing, for connection with a further reaction module's conduit.

29. The service module as claimed in claim 25 further comprising an electrical connector for connecting an electronic device with a power source or a controller.

30. The service module as claimed in claim 25 further comprising a controller to control at least one of the speed of movement of the solid phase, the speed of movement of the fluid phase, a sensor, and a reaction modifying device.

31. A method of performing a solid state synthesis using the system of claim 1 comprising:
passing the mobile solid phase from the first service module to a second reaction module or service module, via the conduit of the first reaction module; and
causing a fluid phase to enter the conduit of the first reaction module through a first input port, flow through the conduit and leave through a first fluid output port.

32. The method of claim 31 further comprising:
providing a second reaction module;
passing the mobile solid phase through a series of the conduits of the reaction modules;
causing a series of independently selected fluid phases to enter the conduits of the reaction modules through the respective fluid input ports, flow through the conduits and leave through the respective fluid output ports to provide a desired polymer attached to a substrate; and
cleaving the polymer from the substrate to provide the desired polymer.

33. The method of claim 32, wherein the polymer is a polypeptide.

34. The method of claim 33, wherein the polypeptide comprises two cysteine moieties and the method further comprises the formation of a sulfur bridge.

35. The method of claim 32, wherein the polymer is a polynucleotide.

36. The method of claim 32 further comprising coupling the polymer with at least one species selected from the group consisting of a polypeptide, a polynucleotide, a sugar, an oligosaccharide, a small molecule, and an antibody.
